(19) United States
(12) Patent Application Publication
(10) Pub. No.: US 2019/0117447 A1
(43) Pub. Date: Apr. 25, 2019

(54) TEMPERATURE CONTROLLING VEST AND METHOD OF MANUFACTURE AND USE FOR RELIEVING OR CONTROLLING MENOPAUSE AND POST-MENOPAUSE SYMPTOMS

(71) Applicant: IMANANO, INC., Lewes, DE (US)

(72) Inventors: Brian Doherty, Aiken, SC (US); Faiz Chowdhury, Irvine, CA (US); David L. ABRAMSON, Alpine, NJ (US)

(21) Appl. No.: 16/091,970

(22) PCT Filed: Apr. 5, 2017
(86) PCT No.: PCT/US17/26246

§ 371 Date: Oct. 5, 2018

Related U.S. Application Data
(60) Provisional application No. 62/318,377, filed on Apr. 5, 2016.

(51) Int. Cl. A61F 7/02

(52) U.S. Cl. CPC A61F 7/02; A61F 2007/0019

(57) ABSTRACT

The field of the invention relates generally to devices and methods for controlling the temperature of a human or non-human body. More specifically, the field of the invention may relate to wearable devices, which may be worn by a subject, which human or non-human, wherein the wearable device is in thermal communication with a portion of the subject's body containing brown adipose tissue and wherein the wearable device comprises a means for providing a constant or near constant temperature to the subject's body and therefore controls the temperature of the subject's body, by using a phase change material. The phase material transitions state at a predetermined temperature which may be higher or lower than the subject's exterior body temperature. The device and method are usable to control temperature-related effects of menopause, post menopause and drug induced thermoregulatory symptoms such as night sweats, hot flashes and sleep disturbance.

### CROSS REFERENCE TO RELATED APPLICATIONS AND INCORPORATION BY REFERENCE

This international patent application, filed under the Patent Cooperation Treaty (PCT), claims benefit of priority to U.S. provisional patent application No. 62/318,377, filed in the United States Patent and Trademark Office (USPTO) on 5 Apr. 2016, titled TEMPERATURE CONTROLLING VEST AND METHOD OF MANUFACTURING AND USE FOR RELIEVING OR CONTROLLING MENOPAUSE AND POST-MENOPAUSE SYMPTOMS, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

### 1. Field of the Invention

The field of the invention relates generally to devices and methods for controlling the temperature of a human or non-human body. More specifically, the field of the invention relates to wearable devices, which may be worn by a subject, human or non-human, wherein the wearable device is in thermal communication with a portion of the subject's body, such as for example, a portion of the torso, and wherein the wearable device comprises a means for providing a constant or near constant temperature to the subject's body and therefore controls the temperature of the subject's body. The means for maintaining constant or near constant temperature may be a phase change material. The area of the person's body may contain brown adipose tissue. The phase material may transition state at a predetermined temperature which may be higher or lower than the subject's exterior body temperature, providing management of the subject's body temperature in the area in thermal communication with the phase change material. The field of the invention also relates to methods and devices for controlling symptoms related to menopause, post menopause, drug induced or other thermoregulatory symptoms such as hot flashes, night sweats and sleep disturbance.

### 2. Background Art

Devices for controlling the temperature of the human body have historically comprised fluid motivating pumps which circulate a cool fluid through a jacket or similar structure that is located in proximity to and is in thermal communication with a subject's body. These systems are complex, expensive, and prone to failure as they necessarily comprise fluid pumps, tubing, and jackets comprising channels or capillary tubing for fluid communication. Furthermore these systems require electrical power in the form of house current or batteries. For all the above reasons such systems are expensive, prone to failure, and may not be the stated objective due to their shortcomings. Furthermore, these systems are not portable. Thus they may require the wearer to remain in the specific location with access to electric power.

Furthermore, it has long been known that female patients may experience certain physical sensations secondary to the onset of menopause. These physical sensations may include, but are not limited to, sudden sensation of increased temperature on the skin or internal to the body, commonly called “hot flashes”, tingling in the fingers, increased heart rate, skin feeling warm and moist to the touch, face becoming flushed, and sweating. These symptoms, which are generally considered to be caused by hormonal changes in the body that result from the normal process of aging, may be predictable as to timing, or they may come on suddenly without any warning or determined periodicity. Likewise, male patients may experience thermoregulatory symptoms such as sleep disturbance, hot flashes, night sweats and other symptoms secondary to treatments for certain conditions, such as drug therapy using, for example, Lupron or Tamoxifen to treat male patients with prostate cancer. All of these symptom, and any symptoms of thermoregulatory nature, are herein referred to as thermoregulatory symptoms.

The above described symptoms of menopause, which may persist long after menopause has technically ended, or in some post-menopause, have been the subject of attempts for relief by various means. One often use menopause relief technique is the application of medications comprising estrogen, such as the well-known drug Premarin®. However, the estrogen drugs prescribed for relief of menopause systems may come with serious medical side effects such as increased risk of stroke, blood clots, heart attacks or cancer of the uterus. The risk of these very serious side effects prevent many women from choosing to utilize estrogen as a means for controlling the above described symptoms of menopause or post-menopause.

Other suggestions for control and or relief of menopause or postmenopausal symptoms include dressing in layers so as to easily be able to control the thermal insulation around a person's body, natural or homeopathic remedies, sipping ice water or simply wearing little or no clothing. However each of these techniques for controlling or relieving menopause or postmenopausal symptoms come with side effects. Dressing in layers and wearing little or no clothing may not be practical in many social situations. Sipping ice water may not always be practical and is certainly not possible while one is asleep or disposed in a situation where no ice water is available. Consuming natural or homeopathic remedies may have undesired physiological side effects. For example, Black cohosh (Actaea racemosa, Cimicifuga racemosa) has been suggested as possibly relieving symptoms. However, it should not be taken if a subject has a liver disorder. Red clover (Trifolium pratense) may increase the chance of bleeding. Dong quai (Angelica sinensis) interacts with common blood thinners, such as Warfarin. Evening primrose oil (Oenothera biennis) may affect blood thinners and some psychiatric medications. Soy can cause mild stomach aches, constipation, and diarrhea.

What is needed in the art therefore is an apparatus and method for controlling the symptoms of menopause, post menopause, drug induced or other thermoregulatory symptoms that do not expose a subject to unwanted physiological side effects, is easily portable, is usable in most if not all social environments, is safe and is effective in controlling the above described symptoms.

### BRIEF SUMMARY OF THE INVENTION

The present invention comprises an apparatus and method that have one or more of the following features and/or steps, which alone or in any combination may comprise patentable subject matter.

One object of the invention is to provide a wearable device operable to control the body temperature of a subject, and, preferably, to control the body temperature of a subject in the area of the subject's body containing brown adipose tissue. Still further, in an embodiment, an object of the invention is to reduce the body temperature of a wearer to a predetermined temperature without causing damage to skin tissue arising from the effects of reducing the skin temperature of the wearer.

Another object of the invention is to reduce the symptoms of menopause, post-menopause, drug induced and other thermoregulatory symptoms; more specifically, to reduce body temperature fluctuations in the wearer during periods of time when the temperature controlling vest of the invention is worn, and preferably also during periods of time when the temperature controlling vest of the invention is not worn.

Another object of the invention is to provide a protocol for the use of a temperature controlling vest for the purpose of reducing body temperature fluctuations in the wearer caused by menopause, post-menopause, drug induced and other thermoregulatory symptoms, or other physiological or environmental conditions.

In accordance with one embodiment of the present invention, the invention comprises a wearable vest, wherein the wearable vest covers a portion of the torso of wearer. In an embodiment the wearable vest may cover a portion of the upper chest, upper back and a portion of the region between the acromion and the neck of the wearer, bilaterally. The wearable vest may, in yet a further embodiment, cover a portion of the neck or shoulders, or both, of the wearer.

The present method and device of the invention overcome the shortcomings of the prior art and that they are operable to control body temperature of a wearer, they have been demonstrated to control and/or manage the symptoms of menopause, post-menopause, drug induced and other thermoregulatory symptoms, or other physiological or environmental conditions and to manage skin temperature, and they have no adverse physiological side effects nor do they damage the skin of the wearer. The device and method of the invention are specifically designed such that they may be utilized for long periods of time at temperatures above that which caused damage to the skin of the wearer.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 depicts a rear view of an embodiment of a wearable temperature controlling vest of the invention, depicting the cells of the invention which contain phase change materials selected to change phase between solid and liquid states a predetermined temperature.

FIG. 2 depicts a cross-section view of a cell of an embodiment of a wearable temperature control and best of the invention, depicting a phase change material captured within the cell and further showing transfer of heat energy to or from the body of the wearer, causing the body temperature of the wearer to change.

FIG. 3 depicts a front view of an embodiment of a wearable temperature controlling vest of the invention, showing the removable straps of the invention, the neck opening, and exemplary positions and shapes of the cells containing the phase change material.

FIG. 4A depicts a rear view of a subject wearing an embodiment of the wearable temperature controlling the rest of the invention.

FIG. 4B depicts a front view of a subject wearing an embodiment of the wearable temperature controlling the rest of the invention, showing the torso straps of the invention encircling the lower torso of the wearer and being removably attached to one another, and further showing the front closing strap secured in place using, for example, hook and loop fasteners.

FIG. 5A depicts a front view of an embodiment of a wearable temperature controlling vest of the invention.

FIG. 5B depicts a front view of an embodiment of a wearable temperature controlling vest of the invention.

FIG. 5C depicts a front view of an embodiment of a wearable temperature controlling vest of the invention.

In the figures, like callout number refer to like features.

### DETAILED DESCRIPTION OF THE INVENTION

The following documentation provides a detailed description of the invention.

All temperature values stated in this application are to be interpreted as referring to degrees Fahrenheit unless otherwise specified.

Although a detailed description as provided in the attachments contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention. Thus the scope of the invention should be determined by the appended claims and their legal equivalents, and not merely by the preferred examples or embodiments given.

As used herein, “phase change material” or “PCM” means a substance or combination of substances with a heat of fusion which, when melting or solidifying at a phase transition, or phase change, temperature T_{PC} enables the substance to absorb or release amounts of heat energy. Heat is absorbed or released when the substance changes state as from from solid to liquid, and vice versa, at the phase transition, or phase change, temperature T_{PC}. T_{PC} is a function of the physical properties of the phase change material. Phase change materials may comprise eutectic materials, salt hydrates, or organic materials. Eutectic materials tend to be solutions of salts in water that have a phase change temperature below 0° C. (32° F.). Salt hydrates are specific salts that are able to incorporate water of crystallization during their freezing process and tend to change phase above 0° C. (32° F.). Organic materials used as PCMs tend to be polymers with long chain molecules composed primarily of carbon and hydrogen. They tend to exhibit high orders of crystallinity when freezing and mostly change phase above 0° C. (32° F.). Examples of materials used as positive temperature organic PCMs include waxes, oils, fatty acids and polyglycols. The definition of “phase change material” or “PCM” further includes compositions comprised of more than one such substance, i.e., mixtures of phase change materials. Examples of substances which are phase change materials are shown in Table 1.

TABLE 1
EXEMPLARY PHASE CHANGE MATERIALS

1-Cyclohexylooctadecane
2-Heptadecanone
3-Heptadecanone
4-Heptadacanone
9-Heptadecanone
Acetamide
Acetic acid
Acrylic acid
Actanilide
Alpha napthol
Aluminium
Azobenzene
Bee wax
Bees wax
Benzamide
Benzoic acid
Benzylamine
Bromcamphor
Camphene
Camphenilone
Capric acid
Caprilic acid
Caprylone
Catechol
Cetyl acid
Chloroacetic acid
Copper
Cyanamide
Dinto toluent (2,4)
Diphenyl amine
Docasyl bromide
Durene
Eladic acid
Formic acid
Glautaric acid
Glycerin
Glycolic acid
Glyolic acid
Gold
Heptadecanone
Heptaudecanoic acid
Hydrocinnamic acid
Hypophosphoric acid
Iron
KNO3
KNO3 (10%)/NaNO3
KNO3/KBr (4.7%)/KCl (7.3%)
KNO3/KCl (4.5%)
KOH
Lauric acid
Lead
Lithium
Methly brombenzoate
Methyl behenate
Methyl eicosanate
Methyl fumarate
Methyl palmitate
Mn (NO₃)₂•6H₂O + MnCl₂•4H₂O (4% w/w)
Myristic acid^{[35]}
Na₂SiO₃•5H₂O
NaCl (26.8%)/NaOH
NaCl (42.5%)/KCl (20.5)/MgCl2
NaCl (5.0%)/NaNO3
NaCl (5.7%)/NaNO3 (85.5%)/Na2SO4
NaCl/NaNO3 (5.0%)
NaCl/KCL (32.4%)/LiCl (32.8%)
NaCl•Na₂SO₄•10H₂O
NaNO2
NaNO3
NaOH
NaOH/Na2CO3 (7.2%)
Nitro napthalene
O-Nitroaniline
O-Xylene dichloride
Oxolate
p-Bromophenol
p-Dichlorobenzene
p-Joluidine
p-Lattic acid
p-Xylene dichloride
Palmatic acid
Paraffin 14-Carbons
Paraffin 15-Carbons
Paraffin 16-Carbons
Paraffin 17-Carbons
Paraffin 18-Carbons
Paraffin 19-Carbons
Paraffin 20-Carbons
Paraffin 21-Carbons
Paraffin 22-Carbons
Paraffin 23-Carbons
Paraffin 24-Carbons
Paraffin 25-Carbons
Paraffin 26-Carbons
Paraffin 27-Carbons
Paraffin 28-Carbons
Paraffin 29-Carbons
Paraffin 30-Carbons
Paraffin 31-Carbons
Paraffin 32-Carbons
Paraffin 33-Carbons
Paraffin 34-Carbons
Pentadecanoic acid
Phenol
Phenylacetic acid
Polyethylene glycol 600
Quinone
Silver
Sodium sulfate (Na₂SO₄•10H₂O)
Stearic acid
Stibene
Succinic anhydride
Thiosinamine
Thymol
Titanium
TME (63% w/w) + H₂O (37% w/w)
Trimyristin
Tristearin
Water
Zinc
α-Chloroacetic acid
α-Nepthylamine
β-Chloroacetic acid

As used herein, “plastic” includes within its meaning a material consisting of any of a wide range of synthetic or semi-synthetic organics that are malleable and can be molded into solid objects of diverse shapes. Plastics are typically organic polymers of high molecular mass, but they often contain other substances. They are usually synthetic, most commonly derived from petrochemicals, but many are partially natural. Examples of plastics include but are not limited to polyester (PES); polyethylene terephthalate (PET); polyethylene (PE); high-density polyethylene (HDPE); polyvinyl chloride (PVC); polyvinylidene chloride (PVDC) (Saran); low-density polyethylene (LDPE); polypropylene (PP); polystyrene (PS); high impact polystyrene (HIPS); polyamides (PA) (Nylons); acrylonitrile butadiene styrene (ABS); polyethylene/Acrylonitrile Butadiene Styrene (PE/ABS); polycarbonate (PC); polycarbonate/Acrylonitrile Butadiene Styrene (PC/ABS); polyurethanes (PU); maleimide/Bismaleimide; melamine formaldehyde (MF); plastarch material; phenolics (PF) or (phenol formaldehydes); polyepoxide (Epoxy); polyetheretherketone (PEEK); polyetherimide (PEI) (Ultem); polyimide; polylactic acid (PLA); polymethyl methacrylate (PMMA) (Acrylic); polytetrafluoroethylene (PTFE); urea-formaldehyde (UF); furan; silicone; and polysulfone, or any combination of these materials.

As used herein, “brown adipose tissue”, “brown adipose fat”, “BAT”, BATF″ and “brown fat” all have the same meaning. In additional to the meaning given these terms as is known in the art, these terms include within their meaning fat tissue that contains many small lipid droplets and a high number of iron-containing mitochondria, is at least partially characterized as burning energy and glucose to make heat.

As used herein, “° F.” shall mean degrees Fahrenheit.

As used herein, “wearer”, “subject”, “person” and “user” mean a person who is wearing a temperature controlling vest of the invention.

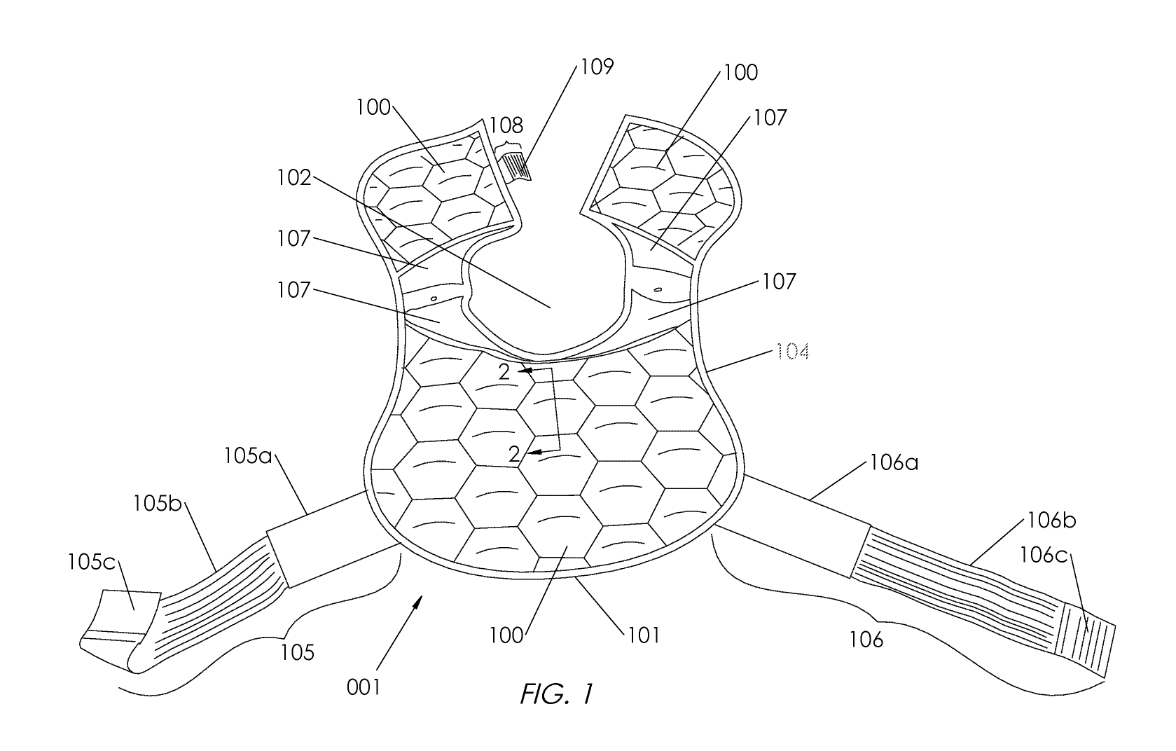

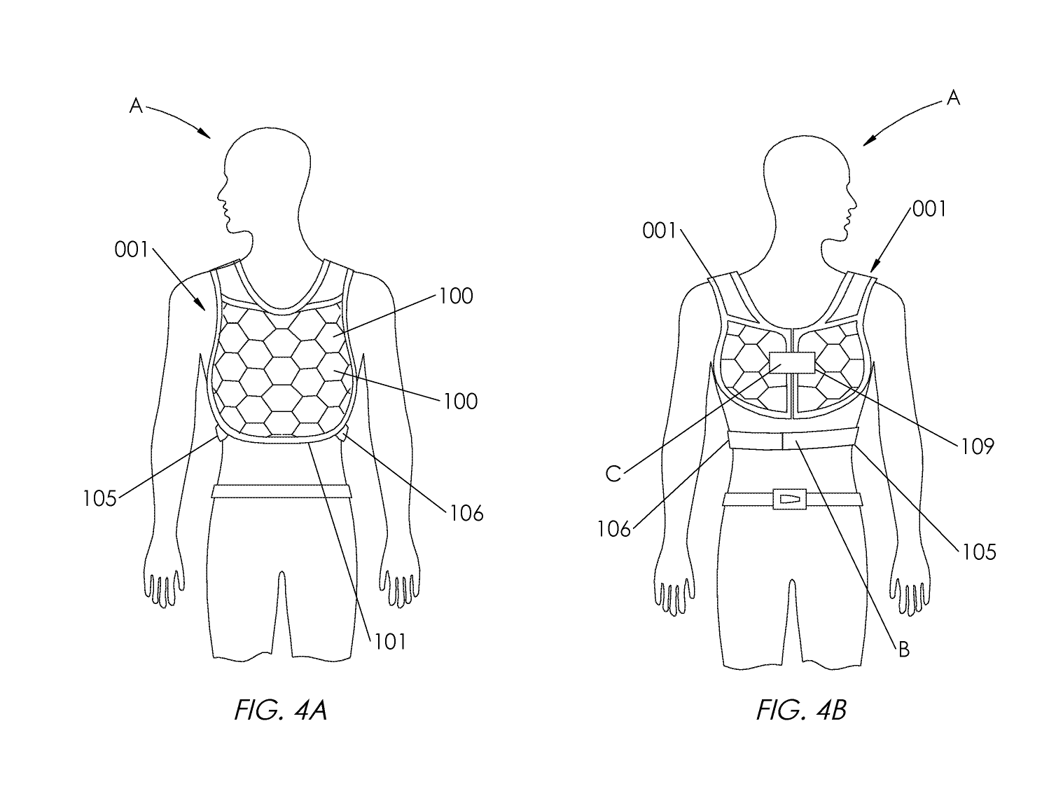

Referring now to FIG. 1, a rear view of an exemplary embodiment of a wearable temperature controlling vest of the invention 001, depicting the cells 100 of the invention which contain phase change materials selected to change phase between solid and liquid states that a predetermined temperature T_{PC} is depicted. The exemplary embodiment depicted in FIG. 1 is a vest shape, and comprises an opening for the wearer's head 102, a seam extending around the periphery of the vest 104; at least one, and preferably a plurality of cells 100 which form enclosed volumes containing PCM material, each cell enclosed by a periphery, a front closure strap 108 comprising a hook fastener forming one part of a hook and loop fastener system. In the example shown, cells 100 take a hexagonal outline, but this is an exemplary embodiment. Cells 100 may take any outline shape desired such as circular, square, rectangular, pentagonal, or any other shape, defined or undefined. Cells 107 also may contain PCM may be of any number or outline shape desired. The vest of the invention 001 may also comprise straps 105 and 106 attached to vest body 101 for wrapping around a portion of the wearer's body, such as for example the lower torso of the wearer, and securing to one another as a means for securing vest 001 to the wearer's body. Straps 105 and 106 may comprises part 105a and 105b, or 106a and 106b, respectively, all of which may be comprised of non-elastic or elastic materials or combinations of such materials. Straps 105 and 106 may also comprise hook and loop components 106c and 105c for fastening together when straps 105 and 106 are extended so as to encircle the wearer's body as depicted further in FIG. 4A and FIG. 4B.

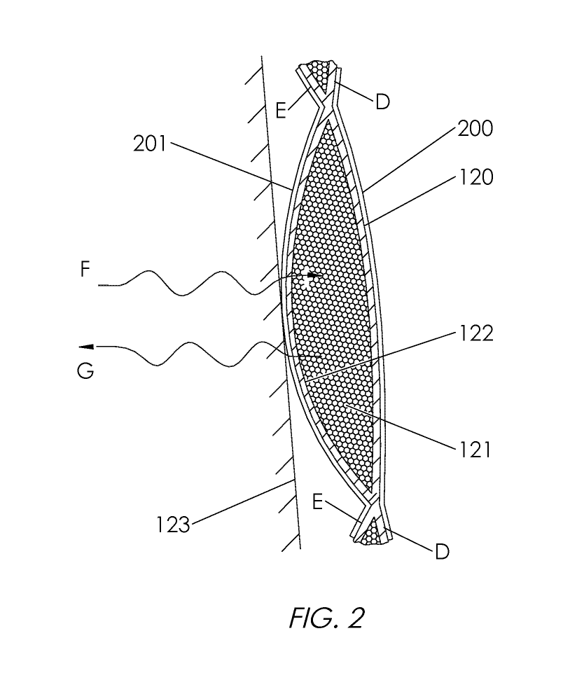

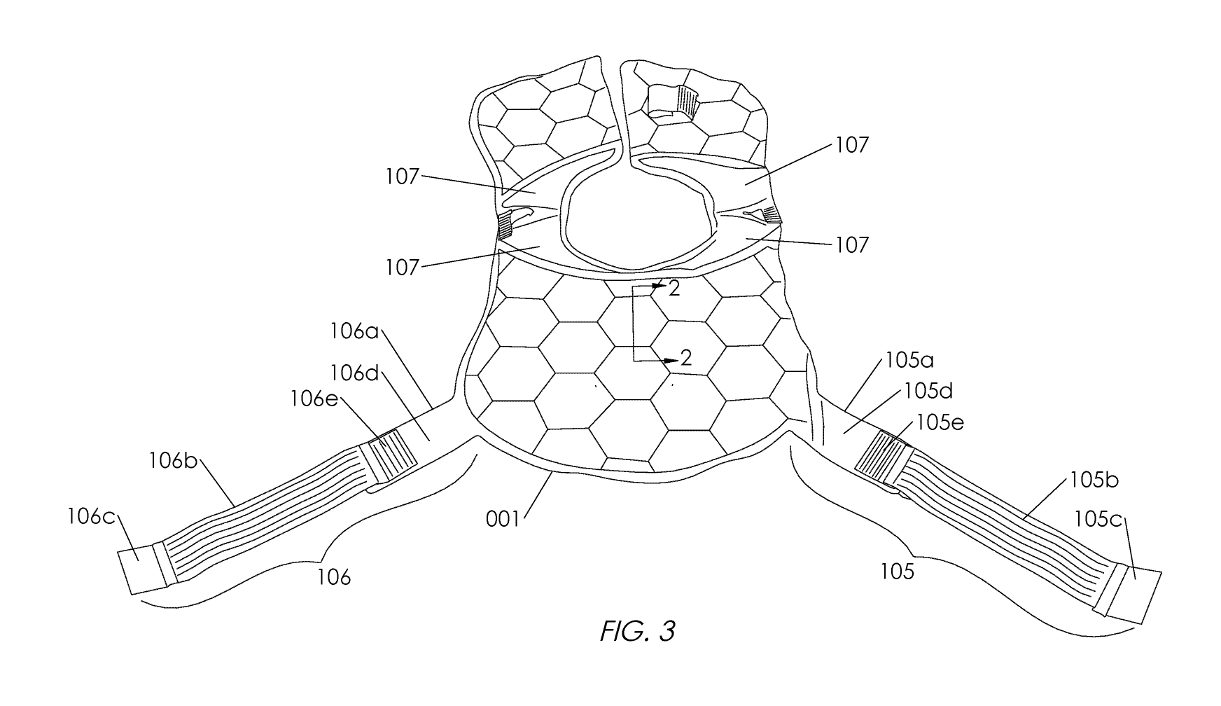

Referring now to FIG. 2, a cross-section view of an exemplary cell 100 of an embodiment of a wearable temperature control and best of the invention, depicting a phase change material captured within the cell and further showing transfer of heat energy to or from the body of the wearer, causing the body temperature of the wearer to change, is depicted. Phase change material 121 is captured within an enclosed volume of cell 100. The enclosed volume may be formed, for example, by an exterior layer 120 and an interior layer 122, wherein the exterior layer 120 and interior layer 122 are cut to shape as desired (for example as depicted in FIGS. 1 and 3), overlaid on one another, and attached together to form at least one cell 100, but preferably a plurality of cells 100 each cell 100 having an enclosed volume for containing phase change material 121. The attachment of exterior layer 120 to interior layer 122 may be by any means known in the art such as, for example, chemical bonding, heat sealing, ultrasonic welding, or the like. The cells thus formed are further defined as having an enclosed volume which contains a PCM. The PCM may be any PCM but is preferably selected by the method of the invention, taught herein. The cells 100, which may each take any outline such as, but not limited to, the hexagonal outline depicted in FIGS. 1 and 3, may be fabricated from any set of materials that are capable of attaching to one another and to form an enclosed volume capable of containing a PCM.

Still referring to FIG. 2, in an embodiment, exterior layer 120 may comprise an inner layer of polyurethane sheet material and an outer layer of loop fastener material for use as a component of hook and loop fastener systems, these two layers being attached together. The outer layer may be, as an example, 100% nylon fabric with polyester foam core and nylon tricot backing such as the product VELTEX® produced by Velcro Industries B.V. CORPORATION NETHERLANDS 22-24 CASTORWEG CURACAO NETHERLANDS ANTIL. Still referring to exterior layer 120, the inner layer may be any thickness of pliable sealable material, such as any pliable plastic material, but in a preferred exemplary embodiment may be 0.014 inch thick polyurethane sheet. Likewise, interior layer 122 may be any thickness of pliable sealable material, such as any pliable plastic material, but in a preferred exemplary embodiment may be 0.014 inch thick polyurethane sheet. The inner layer and outer layer of exterior layer 120 may be attached by any means known in the art, such as, for example, chemical bonding. The vest of the invention 001 may be fabricated by cutting (which may be any form of cutting such as die cutting) or otherwise shaping each layer to the desired shape outline depicted in FIGS. 1 and 3, and as shown being worn by a user in FIGS. 4A and 4B. Once the layers are cut to shape, the seams 104 and seams forming the periphery between the cells 100 of vest 001 may be formed by applying pressure and heat, by chemical bonding, or by any other means known in the art. Thus each cell is separated by other cells by the attachment of interior layer 122 to the inner layer of exterior layer 120 along the periphery of each cell 100 by applying heat and pressure, chemical bonding or any other known means for attaching these layers. In an embodiment, each cell comprises an enclosed volume that is formed by a polyurethane interior layer 122 sealed to the inner layer of exterior layer 120 by applying heat and pressure, such as may be applied by a die pressing these two layers together at the desired seam, or cell periphery, areas. The seam areas separating the cells 100 forming the periphery of each cell 100 extend continuously around the periphery of each cell forming an enclosed volume capable of containing a PCM 121 preventing PCM 121 from escaping the enclosed volume of the cell. In use, a external surface of interior layer 122 is in thermal communication with a surface of a user's body 123 for the purpose of transferring heat energy between the PCM 121 and the user's body in either direction, i.e. cooling user body surface 123 by transferring heat energy from the user's body from body surface 123 to PCM 121 as shown by arrow F, or warming a user's body surface 123 by transferring heat energy from PCM 121 to the user's body as shown by arrow G. Heat energy transfer occurs through contact of a surface of interior layer 122 with user body surface 123 as the PCM changes phase, or physical state.

Thus, in an embodiment, a temperature controlling vest of the invention 001 may be fabricated, having an interior surface comprising interior layer 122 of polyurethane and an exterior surface of 100% nylon fabric with polyester foam core and nylon tricot backing such as the product VELTEX® produced by Velcro Industries B.V. CORPORATION NETHERLANDS 22-24 CASTORWEG CURACAO NETHERLANDS ANTIL forming the loop portion of a hook and loop fastener system. The vest may comprise a plurality of cells 100, each cell 100 forming an interior volume capable of containing a PCM 121 such that the PCM 121 is prevented from escaping the enclosed volume of the cell.

Still referring to FIG. 2, in any embodiment of the invention, an optional layer of graphene 201 may be applied to the external surface of layer 122 on its exterior surface E such that it to aid thermal conductivity between layer 122 and the user body surface 123. Likewise, an optional layer of graphene 200 may be applied to the external surface D of layer 120 to provide thermal insulation between the PCM 121 and the atmosphere surrounding layer 120 in order to prevent undesired heat transfer from PCM 121 to the external atmosphere or environment.

Still referring to FIG. 2, typically, prior to use in a case where it is desired to reduce the temperature of the body of a wearer, the vest of the invention 001 is “charged” to a temperature below a predetermined selected PCM phase transition temperature T_{PC}. The predetermined PCM phase transition temperature T_{PC} may be a temperature at which, given the temperature differential between the temperature of the wearer's body and the PCM caused by the thermal loss of the intervening material layers between the body of the wearer and the PCM, which includes all intervening materials (including clothing worn by the wearer), presents a desired temperature T to the wearer's body while the PCM is transitioning states. For example, if it is desired to apply a temperature T of 49° F. to the body of a wearer, the thermal losses (and thus the temperature differential) between the patient's body and the PCM may be calculated using the known thermal properties of the intervening materials. In the example given, assume a three degree (3°) F. drop in temperature between the body of the wearer and the PCM. Thus it would be desired that the PCM contained within the cells transition phase at a T_{PC} of 46° F., and a suitable PCM or combination of PCMs exhibiting this phase transition temperature T_{PC} is selected. The vest 001 may then be charged to a temperature below T_{PC} 46° F., causing the PCM material 100 in vest 001 to freeze. The vest 001 may then be worn by the wearer to reduce the wearer's body temperature. As the vest 001 warms to T_{PC} 46° F., it will absorb heat F from the body of the wearer as the PCM transitions state from solid to liquid, and will continue to absorb this heat for as long as the PCM continues to transition physical state from solid to liquid. Thus, a constant desired temperature of T 49° F., or any other temperature T desired as determined by the selection of PCM or mixture of PCM comprising the invention, is applied to the wearer's body for as long as the PCM continues to change state.

The length of time required for the PCM 121 to change state is a function of the volume of PCM 121 contained with the cells and the temperature differential between the PCM 121 and wearer's body. The larger the temperature differential, the faster heat will transition from a surface of the wearer's body 123 to the PCM 121 and the faster the PCM 121 will transition state. Also, the larger the volume of PCM 121 material contained within each cell, the longer it will take, in terms of time, for the PCM 121 to change state. Additionally, the thickness of each cell and thus the flexibility and comfort of the vest 001 of the invention are affected by the volume of PCM 121 disposed in the cell. When the volume of PCM 121 in a cell is increased, the flexibility of the vest is reduced, and vice versa. Methods of the invention for determining the PCM 121 to be used in the invention, and for optimizing the volume of PCM 121 contained with a cell are to 1) determining a desired temperature T to be applied to the body of a wearer; 2) determining the temperature drop ΔT between the body of the wearer and PCM 121 taking into account all intervening materials; 3) determining the desired temperature T_{PC} at which the PCM 121 must change state in order to apply the desired temperature T to the body of a wearer by adding ΔT to T; and 4) selecting a PCM 121 or combination of PCMs 121 that will transition state at the desired PCM 121 transition temperature T_{PC} to be disposed within the cells of the invention; further, optionally, the volume of PCM 121 to be disposed within a cell of a vest of the invention may be determined by 5) determining a desired flexibility of the vest; 6) determining the volume of the specific PCM 121 selected that is required to achieve the minimum flexibility of the vest. Step 6) may be carried out by trial and error by producing a prototype vest 001 with a specific volume of PCM 121 disposed within the vest and then increasing or decreasing the volume of PCM 121 disposed within the cells of the invention until the volume of PCM 121 in the cells to achieve the desired flexibility of the vest is achieved.

The above described process may also be reversed such that the wearer's body 123 absorbs heat from the PCM 121 as depicted by G in FIG. 2. In such a case, the steps of the above process are reversed, and the PCM is selected to be characterized by a T_{PC} that is higher than the desired temperature T to be applied to the wearer's body, such that T+ΔT=T_{PC}.

In the above manner, PCM 121 is in thermal communication with surface of a body of a subject 123. In embodiments of the methods described below, the surface of the subject's body 123 is in an area of the body containing brown adipose tissue such that the wearing of the vest by a subject causes a desired temperature change in the brown adipose tissue of the subject.

Referring now to FIG. 3, a front view of an embodiment of a wearable temperature controlling vest of the invention 001, showing the removable straps of an embodiment the invention, the neck opening, and exemplary positions and shapes of the cells containing the phase change material is depicted. The exemplary embodiment depicted in FIG. 3 is a vest shape, and comprises a neck opening for the wearer's head 102, a seam extending around the periphery of the vest 104; cells 100 which have peripheries forming enclosed volumes containing PCM material. In the example shown, cells 100 take a hexagonal outline, but this is merely an exemplary embodiment. Cells 100 may take any outline shape desired such as circular, square, rectangular, pentagonal, or undefined. Cells 107 which also may contain PCM may be of any number or outline shape desired. The vest of the invention 001 may also comprise straps 105 and 106 attached to vest body 101 for wrapping around a portion of the wearer's body, such as for example the lower torso of the wearer, and securing to one another as a means for securing vest 001 to the wearer's body. Straps 105 and 106 may comprise parts 105a and 105b, or 106a and 106b, respectively, all of which may be comprised of non-elastic or elastic materials or combinations of such materials. Straps 105 and 106 may also comprise hook and loop components 106c, 106d and 106e and 105c, 105d and 105e for fastening together when straps 105 and 106 are extended so as to encircle the wearer's body as depicted further in FIG. 4A and FIG. 4B.

Referring now to FIG. 4A, a rear view of a subject A wearing an embodiment of the wearable temperature controlling vest 001 of the invention is depicted. A second portion of the vest 001 covers a rear portion of the upper torso of user A. Cells 100 contain PCM that, in an embodiment, has been selected by a method of the invention. Straps 105 and 106 extend from attachment points to vest 001 around to the front side of the wearer A, where they are attached together by any means known in the art, for example hook and loop fasteners.

Referring now to FIG. 4B, a front view of a subject wearing an embodiment of the wearable temperature controlling the rest of the invention, showing the torso straps of the invention encircling the lower torso of the wearer and being removably attached to one another, and further showing the front closing strap secured in place using, for example, hook and loop fasteners, is depicted. A first portion of the vest 001 covers a front portion of the torso of user A. Cells 100 contain PCM that, in an embodiment, has been selected by the method of the invention. Straps 105 and 106 extend from attachment points to vest 001 around to the front side of the wearer A, where they are attached together at area B by any means known in the art, for example hook and loop fasteners. Strap 109, which may comprise the hook portion of a hook and loop fastener system, may operate to connect front halves of vest 001 when portions of strap 109 are placed in contact at point C with the exterior surface of exterior layer 120 which may be comprised of material forming the loop portion of a hook and loop fastener system. Thus, the removable attachment of straps 105 and 106 to one another and the attachment of strap 109 to the front halves of vest 001 operate to secure the wearable temperature controlling vest of the invention to the user's body.

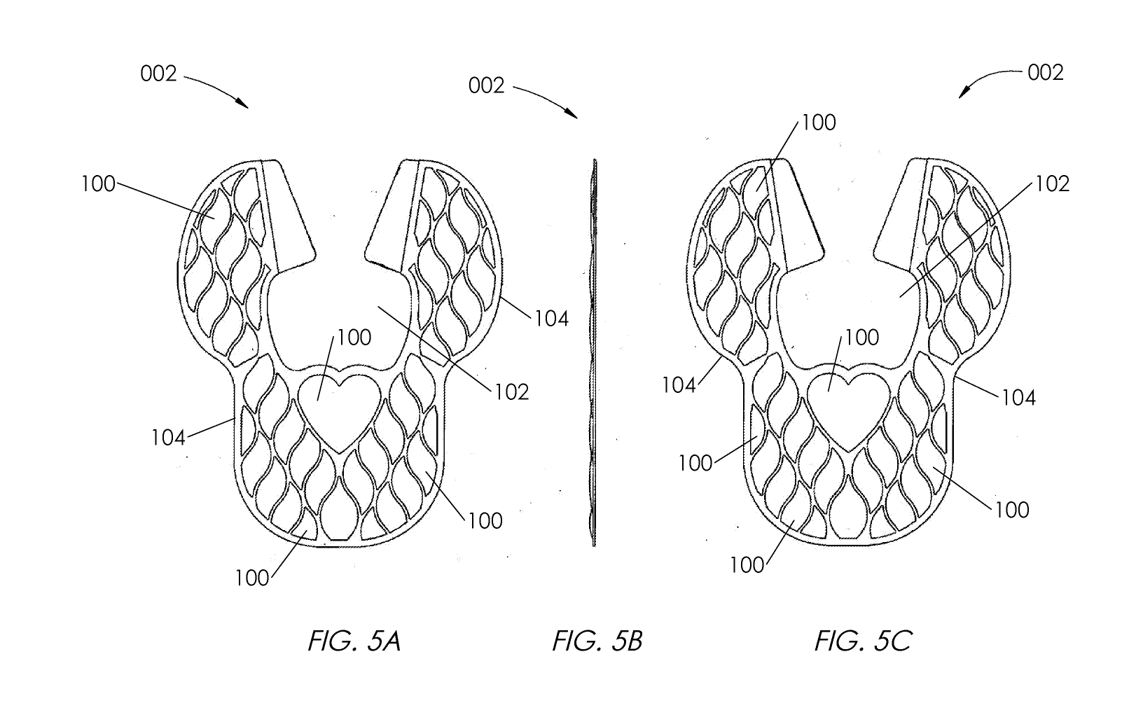

Referring now to FIG. 5A a front view of an alternate embodiment of a wearable temperature controlling vest of the invention 002, showing exemplary positions and shapes of the cells containing the phase change material is depicted. The exemplary embodiment depicted in FIG. 5A is first portion formed in a bolero shape, and comprises an opening for the wearer's head 102, a seam extending around the periphery of the vest 104; cells 100 which form enclosed volumes containing PCM material, and a rear closure element comprising a hook and loop fastener system. In the example shown, cells 100 are depicted as comprising a specific outline, but this is an exemplary embodiment. Cells 100 may take any outline shape desired such as circular, square, rectangular, pentagonal, arbitrary, defined or undefined. A user may place their head through opening 102 such that opening 102 partially surrounds the user's neck, allowing the cells 100 to lay against a front portion of the subject's upper torso, a rear portion of the subject's torso, and, in embodiment, a portion of the subject's shoulder between the shoulder socket and the neck. The vest may be secured there by means of hook and loop fasteners located on tabs located adjacent to opening 102. FIG. 5B depicts a side view of this exemplary embodiment, and FIG. 5C depicts a rear view of the embodiment.

In alternate embodiments of the method, more than one PCM may comprise the PCM utilized in the invention, resulting in the use of a PCM mixture, as may be necessary to achieve a specific desired PCM phase change temperature T_{PC}, or to achieve any desired step function in, or duality of, PCM phase transition temperature T_{PC}. In further alternate embodiments of the invention, one or more covering materials may be utilized, and each may be analyzed for thermal conductivity in order to achieve the desired thermal transfer between the body of a user and the temperature control pad. Still further, in any embodiment of the invention, the PCM material may be mixed with graphene to enhance the PCM thermal transfer properties. In an embodiment, the amount of graphene mixed into the PCM may be up to ten percent graphene to PCM, by weight. In further embodiments, the amount of graphene mixed into the PCM may be higher than ten percent graphene to PCM, by weight.

It is to be understood that the outline shapes and cell shapes of the temperature controlling vest of the invention is depicted as representative shapes 001 and 002 in the figures, and that the scope of the invention encompasses all other shapes, and all other embodiments that are able to be applied to other areas of a subject's body, and especially all other areas of a subject's body in which the use of a temperature controlling apparatus of the invention results in thermal communication between a PCM and brown adipose tissue of a subject. The use of the term “vest” is for convenience only as an aid in referring to the temperature controlling apparatus of the invention and is not be construed as limiting the claimed invention to only vest shapes. Thus the term vest is inclusive of all other shapes.

Use of the temperature controlling vest 001 or 002 of the invention to cool or otherwise modulate the body temperature of the wearer in the area of the wearer's body that is in contact with the vest or near the vest may stimulate brown adipose tissue (BAT) by cooling or warming the user's BAT in the area of the user's body that is in contact or near the temperature controlling vest of the invention, which in turn causes hormonal responses in the wearer's body restuling in a desired thermoregulation of the wearer's body, and thus reducing one or more symptoms such as hot flashes, night sweats, sleep disturbance, tingling in the fingers, increased heart rate, skin feeling warm and moist to the touch, face becoming flushed that may be associated with menopause, aging, chemical use, or drug use that may be related to therapies for treating unrelated medical conditions such as prostate cancer.

Accordingly, the invention further comprises methods for such use. In a general embodiment, the method comprises the steps of determining a desired phase change temperature for the PCM, selecting a PCM that is characterized by a phase change at the selected phase change temperature, and the user wearing a temperature controlling vest comprising the selected PCM in accordance with a protocol thus cooling the user's BAT.

In an embodiment, the invention comprises a method for relief from menopause symptoms such as body temperature swings, hot flashes, fluctuation in skin temperature, and other temperature-related symptoms. In a further embodiment, the invention comprises a method for treating hormonal fluctuation and the symptoms of hormonal fluctuation in male patients that may result from chemical or drug use, such as, for example, the use of drug therapy using Lupron or Tamoxifen to treat prostate cancer. These antiestrogen medications/aromatase inhibitor medications modulate hormonal responses resulting in chemically induced disruptive thermoregulatory symptoms causing, for example, but not limited to, hot flashes and night sweats in men and women. However, when the invention is charged to a temperature below body temperature, for example in the range of T_{PC} of between 48° F. to 65° F., inclusive, and is worn by a user thus cooling the user's brown adipose tissue, there has been shown to be a resulting beneficial effect of reducing these body temperature fluctuation-related symptoms.

In an embodiment, the above symptoms may be relieved or controlled by the lowering of the wearer's body temperature that results from the wearer's wearing a temperature controlling vest of the invention in contact or near a user's BAT, wherein the vest comprises PCM material that is selected by the process of the invention to transition phase at a temperature T_{PC}. While the temperature T_{PC} may be any temperature below 98.6° F., preferred ranges of T_{PC} are 45° F. to 80° F.; 45° F. to 59° F., 60° F. to 69° F.; 70° F. to 80° F.; 48° F. to 65° F. and any range within 45° F. to 80° F.

Exemplary protocols of the method of the invention for treating menopause symptoms in female patients are as follows. In a first protocol, the wearer wears a temperature controlling vest of the invention comprising PCM selected to change state at any temperature identified above for T_{PC} for designated periods during a day. In a second protocol, the wearer wears a temperature controlling vest of the invention comprising PCM selected to change state at any temperature identified above for T_{PC} for a period of thirty (30) minutes prior to breakfast each morning and thirty (30) minutes prior to dinner for a specified period of days. In a third protocol, the wearer wears a temperature controlling vest of the invention comprising PCM selected to change state at any desired temperature or any temperature identified above for T_{PC} for a period of thirty (30) minutes prior to breakfast time and continuing for an additional thirty (30) minutes after the beginning of breakfast time for a total of sixty (60) minutes each morning, and then again thirty (30) minutes prior to dinner time and continuing for an additional thirty (30) minutes after the beginning of dinner time for a total of sixty (60) minutes, all together totaling one hundred twenty (120) minutes each day, for a specified period of days. As used herein, “breakfast time” and “dinner time” may be defined as having their ordinary meaning. Alternatively, “breakfast time”, in an embodiment, is defined as occurring between 6:00 am and 9:00 am, and “dinner time” in an embodiment is defined as occurring between 4:00 pm and 7:00 pm. The terms “breakfast time” and “dinner time” are use solely used to identify the above times and not any activity related to consumption of food. In other words, the consumption of food is not be construed as a limitation in the claims.

What is claimed is:

1. A wearable temperature controlling vest, comprising:
a first portion for covering a portion of a wearer's upper front torso;
a second portion for covering a portion of a wearer's upper rear torso;
said first portion and said second portion connected together, forming a wearable vest;
wherein each of said first portion and said second portion further comprise at least one cell having an enclosed volume; and
a phase change material disposed within said enclosed volume of each cell;
wherein, when said wearable temperature controlling vest is worn by a wearer, said phase change material is in thermal communication with a portion of the subject's brown adipose tissue.

2. The wearable temperature controlling vest of claim 1, wherein said at least one cell is further defined as a plurality of cells.

3. The wearable temperature controlling vest of claim 2, wherein each of said cells is further defined as having a periphery, and wherein said cells are formed from a first layer of polyurethane and a second layer of polyurethane overlaid on one another, wherein said first layer and said second layer are attached by seams formed along each cell periphery by attachment means selected from the group consisting of chemical bonding, heat sealing, and ultrasonic welding along seam lines forming said cells, the attachment creating an enclosed volume within the cell for containing phase change material.

4. The wearable temperature controlling vest of claim 3, wherein said second layer of polyurethane has an exterior surface, and wherein a layer of 100% nylon fabric with polyester foam core and nylon tricot backing for forming the loop portion of a hook and loop fastener system is attached to said exterior surface.

5. The wearable temperature controlling vest of claim 1, further comprising an opening for allowing a wearer's neck to pass through said opening when said vest is worn.

6. The wearable temperature controlling vest of claim 2, wherein said vest comprises an opening for allowing a wearer's neck to pass through said opening when said vest is worn.

7. The wearable temperature controlling vest of claim 3, wherein said vest comprises an opening for allowing a wearer's neck to pass through said opening when said vest is worn.

8. The wearable temperature controlling vest of claim 4, wherein said vest comprises an opening for allowing a wearer's neck to pass through said opening when said vest is worn.

9. The wearable temperature controlling vest of any of claims 1-8, further comprising a first strap and second strap attached to said vest, said straps adapted to extend around and encircle a wearer's lower torso when worn.

10. The wearable temperature controlling vest of any of claims 1-8, further comprising a first strap and second strap attached to said vest, said straps adapted to extend around and encircle a wearer's lower torso when worn, said straps releasable attachable by hook and loop fasteners.

11. The wearable temperature controlling vest of any of claims 1-8, wherein said phase change material is selected to change physical state at a temperature between 45° F. and 80° F.

12. The wearable temperature controlling vest of any of claims 1-8, wherein said phase change material is selected to change physical state at a temperature between 45° F. and 59° F.

13. The wearable temperature controlling vest of any of claims 1-8, wherein said phase change material is selected to change physical state at a temperature between 60° F. and 69° F.

14. The wearable temperature controlling vest of any of claims 1-8, wherein said phase change material is selected to change physical state at a temperature between 70° F. and 80° F.

15. The wearable temperature controlling vest of any of claims 1-8, wherein said phase change material is selected to change physical state at a temperature between 48° F. to 65° F.

16. The wearable temperature controlling vest of claim 3, wherein said second layer of polyurethane has an exterior surface, and wherein a layer of graphene is attached to said exterior surface of said second layer of polyurethane.

17. The wearable temperature controlling vest of claim 3, wherein said first layer of polyurethane has an exterior surface, and wherein a layer of graphene is attached to said exterior surface of said first layer of polyurethane.

18. The wearable temperature controlling vest of claim 3, wherein each of said first layer of polyurethane and said second layer of polyurethane has an exterior surface, and wherein a layer of graphene is attached to said exterior surface of said first layer of polyurethane and wherein a layer of graphene is attached to said exterior surface of second layer of polyurethane.

19. A wearable temperature controlling vest, comprising:
a first portion for covering a portion of a wearer's upper front torso, left shoulder, and right shoulder;
wherein said first portion further comprises at least one cell having an enclosed volume; and
a phase change material disposed within said enclosed volume of each cell;
wherein, when said wearable temperature controlling vest is worn by a wearer, said phase change material is in thermal communication with a portion of the subject's brown adipose tissue.

20. The wearable temperature controlling vest of claim 16, wherein said at least one cell is further defined as a plurality of cells.

21. The wearable temperature controlling vest of claim 20, wherein each of said cells is further defined as having a periphery, and wherein said cells are formed from a first layer of polyurethane and a second layer of polyurethane overlaid on one another, wherein said first layer and said second layer are attached by seams formed along each cell periphery by attachment means selected from the group consisting of chemical bonding, heat sealing, and ultrasonic welding along seam lines forming said cells, the attachment creating an enclosed volume within the cell for containing phase change material.

22. The wearable temperature controlling vest of claim 21, wherein said second layer of polyurethane has an exterior surface, and wherein a layer of 100% nylon fabric with polyester foam core and nylon tricot backing for forming the loop portion of a hook and loop fastener system is attached to said exterior surface.

23. The wearable temperature controlling vest of claim 19, further comprising an opening for allowing a wearer's neck to pass through said opening when said vest is worn.

24. The wearable temperature controlling vest of claim 20, wherein said vest comprises an opening for allowing a wearer's neck to pass through said opening when said vest is worn.

25. The wearable temperature controlling vest of claim 21, wherein said vest comprises an opening for allowing a wearer's neck to pass through said opening when said vest is worn.

26. The wearable temperature controlling vest of claim 22, wherein said vest comprises an opening for allowing a wearer's neck to pass through said opening when said vest is worn.

27. The wearable temperature controlling vest of any of claims 19-26, wherein said phase change material is selected to change physical state at a temperature between 45° F. and 80° F.

28. The wearable temperature controlling vest of any of claims 19-26, wherein said phase change material is selected to change physical state at a temperature between 45° F. and 59° F.

29. The wearable temperature controlling vest of any of claims 19-26, wherein said phase change material is selected to change physical state at a temperature between 60° F. and 69° F.

30. The wearable temperature controlling vest of any of claims 19-26, wherein said phase change material is selected to change physical state at a temperature between 70° F. and 80° F.

31. The wearable temperature controlling vest of any of claims 19-26, wherein said phase change material is selected to change physical state at a temperature between 48° F. to 65° F.

32. The wearable temperature controlling vest of any of claims 19-26, wherein said phase change material is mixed with graphene at up to ten percent graphene to phase change material by weight.

33. The wearable temperature controlling vest of claim 21, wherein said second layer of polyurethane has an exterior surface, and wherein a layer of graphene is attached to said exterior surface of said second layer of polyurethane.

34. The wearable temperature controlling vest of claim 21, wherein said first layer of polyurethane has an exterior surface, and wherein a layer of graphene is attached to said exterior surface of said first layer of polyurethane.

35. The wearable temperature controlling vest of claim 21, wherein each of said first layer of polyurethane and said second layer of polyurethane has an exterior surface, and wherein a layer of graphene is attached to said exterior surface of said first layer of polyurethane and wherein a layer of graphene is attached to said exterior surface of second layer of polyurethane.

36. A method for thermally managing an area of a body of a person, comprising the steps of:
providing a temperature controlling vest comprising a plurality of cells, each cell having an enclosed volume containing a phase change material;
placing said temperature controlling vest on the upper torso a person's body such that said phase change material is in thermal communication with an area of a person's body desired to be thermally managed, while said phase change material is undergoing phase transition.

37. The method for thermally managing an area of a body of a person, wherein said area of a person's body desired to be thermally managed is further defined as an area of a person's body that contains brown adipose tissue, and wherein, when said wearable temperature controlling vest is worn by a wearer, said phase change material is in thermal communication with a portion of the subject's brown adipose tissue.

38. The method for thermally managing an area of a body of a person of either of claim 36 or 37, wherein said step of providing a temperature controlling vest is further defined as wherein said phase change material changes physical state at a predetermined phase transition temperature.

39. The method for thermally managing an area of a body of a person of either of claim 36 or 37, wherein said step of providing a temperature controlling vest is further defined as said phase transition predetermined temperature being between 45° F. and 80° F.

40. The method for thermally managing an area of a body of a person of either of claim 36 or 37, wherein said step of providing a temperature controlling vest is further defined as said predetermined phase transition temperature being between 45° F. and 59° F.

41. The method for thermally managing an area of a body of a person of either of claim 36 or 37, wherein said step of providing a phase change material warming pad comprising is further defined as said predetermined phase transition temperature being between 60° F. and 69° F.

42. The method for thermally managing an area of a body of a person of either of claim 36 or 37, wherein said step of providing a phase change material warming pad comprising is further defined as said predetermined phase transition temperature being between 70° F. and 80° F.

43. The method for thermally managing an area of a body of a person of either of claim 36 or 37, wherein said step of providing a phase change material warming pad comprising is further defined as said predetermined phase transition temperature being between 48° F. to 65° F.

44. A method for providing relief from menopause, post menopause, or drug-induced thermoregulatory symptoms such as body temperature swings, hot flashes, fluctuation in skin temperature, sleep disturbance, night sweats and other temperature-related symptoms, comprising:
determining a desired temperature T for applying to an area of a subject's body containing brown adipose tissue;
selecting a phase change material for use in a temperature controlling vest for maintaining a temperature T_{PC} while the phase change material is transitioning physical state, taking into account thermal losses between the phase change material and the subject's body such that heat transferred from the subject's body to the phase change material during said physical state transition results in said desired temperature T being applied to the subject's body;
providing a temperature controlling vest having an plurality of cells, each cell comprising an enclosed volume containing the selected phase change material;
charging the temperature controlling vest comprising the selected phase change material by subjecting the temperature controlling vest to a temperature that is below the phase change material phase transition temperature T_{PC}; and
causing the subject to wear the temperature controlling vest such that the phase change material is in thermal communication with an area of a subject's body containing brown adipose tissue while the phase change material is transitioning state, thereby causing heat to be transferred from the brown adipose tissue to the phase change material.

45. The method for providing relief from menopause or post menopause symptoms such as body temperature swings, hot flashes, fluctuation in skin temperature, and other temperature-related symptoms to a subject of claim 44, further defined as the phase change material phase transition temperature T_{PC} being between 45° F. and 80° F., inclusive.

46. The method for providing relief from menopause or post menopause symptoms such as body temperature swings, hot flashes, fluctuation in skin temperature, and other temperature-related symptoms to a subject of claim 44, further defined as the phase change material phase transition temperature T_{PC} being between 45° F. and 59° F., inclusive.

47. The method for providing relief from menopause or post menopause symptoms such as body temperature swings, hot flashes, fluctuation in skin temperature, and other temperature-related symptoms to a subject of claim 44, further defined as the phase change material phase transition temperature T_{PC} being between 60° F. and 69° F., inclusive.

48. The method for providing relief from menopause or post menopause symptoms such as body temperature swings, hot flashes, fluctuation in skin temperature, and other temperature-related symptoms to a subject of claim 44, further defined as the phase change material phase transition temperature T_{PC} being between 70° F. and 80° F., inclusive.

49. The method for providing relief from menopause or post menopause symptoms such as body temperature swings, hot flashes, fluctuation in skin temperature, and other temperature-related symptoms to a subject of claim 44, further defined as the phase change material phase transition temperature T_{PC} being between 48° F. to 65° F., inclusive.

50. The method of any of claims 44-49, wherein said temperature controlling vest is worn by the wearer for designated periods during each day.

51. The method of any of claims 44-49, wherein said temperature controlling vest is worn by the wearer for a first designated period and a second designated period during each day, each period lasting 60 minutes.

52. The method of any of claims 44-49, wherein said first period occurs during the morning and the second period occurs during the evening.

53. The method of any of claims 44-49, wherein said first period occurs during the morning between 6:00 am and 9:00 am, and the second period occurs during the evening between 4:00 pm and 7:00 pm.

54. The method of any of claims 44-49, wherein said temperature controlling vest is worn by the wearer for designated periods during a day for a period of thirty minutes prior to breakfast and continuing for an additional thirty minutes after the beginning of breakfast for a total of sixty minutes each morning, and then again thirty minutes prior to dinner and continuing for an additional thirty minutes after the beginning of dinner for a total of sixty minutes, all together totaling one hundred twenty minutes each day.
